(19) United States
(12) Patent Application Publication
(10) Pub. No.: US 2015/0272771 A1
(43) Pub. Date: Oct. 1, 2015

(54) Perinatal Care Device with Disposable Cover

(71) Applicant: Paula O'Brien, Bellingham, WA (US)

(72) Inventor: Paula O'Brien, Bellingham, WA (US)

(21) Appl. No.: 14/728,436

(22) Filed: Jun. 2, 2015

Related U.S. Application Data
(63) Continuation-in-part of application No. 13/799,801, filed on Mar. 13, 2013.
(60) Provisional application No. 61/768,767, filed on Feb. 25, 2013.
(60) Provisional application No. 62/144,259, filed on Apr. 7, 2015.

(51) Int. Cl. A61F 5/37; A61F 7/00; A61B 19/08

(52) U.S. Cl. CPC A61F 5/3707; A61B 19/08; A61F 7/00; A61F 2007/0055; A61F 2007/0022; A61F 2007/0002; A61F 2007/0091

(57) ABSTRACT

A customized disposable surgical coversheet for a perinatal care device comprising a shield that is configured to be placed over a patient's lower thorax to provide a cost effective and efficient method of providing skin to skin contact between the mother and baby during and following cesarean section birth. The coversheet will provide high barrier strength and protection with comfort and softness for use with the perinatal care device.

### RELATED APPLICATIONS

This application, (Attorney's Ref. No. P218546) is a continuation-in-park of U.S. patent application Ser. No. 13/799,801 filed Mar. 13, 2013, currently pending.

U.S. application Ser. No. 13/799,801, claims benefit of U.S. Provisional Patent Application Ser. No. 61/768,767 filed Feb. 25, 2013, now expired.

This application, (Attorney's Ref. No. P218546) also claims benefit of U.S. Provisional Patent Application Ser. No. 62/144,259 filed Apr. 7, 2015, currently pending.

The contents of all related applications listed above are incorporated herein by reference.

### BACKGROUND

The invention relates to the perinatal care of mothers and babies. A large body of research supports early skin-to-skin contact as a precursor to successful initiation and duration of breastfeeding and improved well-being of the mother-baby dyad. The American Academy of Pediatrics has recommended that “all healthy infants should be placed and remain in skin-to-skin contact with their mothers immediately after delivery until the first feeding occurs” (Gartner, L. & Eidelman, A., Breastfeeding and the use of human milk, Pediatrics, 115, 496-506. Doi:10:1542/peds. 2004-2491 (2005), p. 498).

However, in the case of cesarean births, this presents practical difficulties. First, the Joint Committee on Administration Rules for Obstetric Departments (2012) recommends a temperature of 72-76° F. (22-24° C.) in the delivery suite to maintain a neutral thermal environment for the newborn. Surgical operating rooms are usually kept at a lower temperature. In the U.S.A., the average operating room temperature is between 68-73° F. (20-23° C.) and sometimes as low as 66-68° F. (19-20° C.), which is undesirably cool for a newborn baby. Second, surgery may continue for a considerable period after the actual delivery of the baby, for example, to repair and close the surgical incision made for the cesarean birth. It may be hazardous to the baby to be exposed too closely to the sharp instruments, hard equipment, rapid activity, and fluids involved in surgery. Third, surgical operating tables are typically narrow, to allow easy access for the surgical personnel, presenting a risk that if the baby slips off the mother it may easily fall to the floor and be harmed. That has in the past required a nurse to be in constant attendance next to the mother if the baby is placed skin to skin with the mother while she is still on the operating table.

### SUMMARY

An embodiment of a perinatal care device provides a shield that is placed over the mother's lower thorax. The shield is generally in the shape of a part dome, with its rim extending towards the mother's head. Warm air is delivered to a manifold in or on the shield, from which it is distributed to the space between the shield and the mother's thorax. Thus, a region of warm air can be provided within which the baby and mother are comfortable, and the baby is physically separated by the shield from any continuing surgical activity on the mother's abdomen.

In an embodiment, jets of warm air from the upper rim of the shield are angled obliquely, inwards and towards the mother's head. Thus, the region of warm air can extend headward further than the shield. The shield thus does not need to extend over the mother's head to a point that would cause a feeling of claustrophobia in the average patient.

In an embodiment, the shield is provided with wings or upper segment, extending headward towards the mother's shoulders, and sufficiently high that, if the mother loses her grip on the baby, the baby will be retained by the wings, and does not risk falling off the operating table. In a further embodiment, the wings or upper segment are part of a continuous wall extending round above the mother's head. The ends of the wings are thus connected, increasing the strength and stiffness of the wings with only a small increase in the weight of the device. The wall may extend round a pillow or wedge raising the mother's head, or head and upper torso, so that the wall does not uncomfortably enclose the mother's face.

The device may be constructed with a center section that lies on the operating table, or on a mattress, under the mother's body, and is held in place largely by the mother's weight, and two side sections that form the shield and (if present) the wings. To form the shield, the two side sections may be folded together over the mother's body and joined in the middle by clips, clasps, snap fasteners, or any other suitable mechanism. The fasteners may be adjustable to fit mothers of different sizes, either by overlapping the side sections in the middle for a smaller mother, or allowing a gap between the fasteners for a larger mother, or both.

The manifold to distribute warm air may be a separate liner that fits inside the shield, and is held in place by allowing it to expand under the pressure of the air in the manifold. The liner can then bridge a gap at the join between two halves of the shield.

The shield may be an inflatable device, supported by the pressure of air in compartments inside it. The shield can then be light in weight, and very compact, for ease of storage and transport, in its uninflated condition. The supporting compartments for the shield may be the air chambers of the warm air manifold, separate chambers connected to the same or a different air supply, or separate chambers that are inflated and then closed off. The shield may be inflated over the patient's body, after it has been positioned and at least partially assembled. The inflatable shield may comprise a framework of inflatable tubes with non-inflatable sheets or membranes filling in the spaces between the tubes.

Various parts of the device may be made reusable, in which case they should be easy to clean and sterilize, or may be made disposable. For example, where the shield and the liner are separate components, the shield may be made reusable, and may be relatively robust, providing the main mechanical strength. The liner with the warm air manifold may cover the inside of the shield, protecting the shield from contamination. The liner may be disposable. The liner can then be of relatively light construction, because it is mechanically supported by the shield when in use.

The perinatal care device may be combined with various other devices. For example, a ramp may be provided to elevate the mother's head and upper torso, which may be beneficial to assist the mother's comfort and breathing. A “left lateral tilt” support may be provided under the right side of the mother's torso. The patient may lie on an inflatable air transfer bed, such as those sold by Airpal, Inc., of Coopersburg, Pa., U.S.A. Any of those other devices may also be inflatable, and may be inflated from the same air supply as the perinatal care device. The various devices may be integrated into a unitary construction, or may be provided with snaps or other attachments to secure them together releasably.

An electrosurgical grounding pad, such as those sold by Megadyne Medical Products, Inc., Draper, Utah, U.S.A., may be provided. It has been found experimentally that the electrosurgical grounding pad can be placed under the air transfer bed, provided the air transfer bed is not inflated until electrosurgery has been completed. The mentioned ramp does not interfere with electrosurgery, because the mother's lower torso and legs, which are not elevated by the ramp, can provide sufficient connection with the electrosurgical grounding pad. A commercially available warming pad may be placed under the electrosurgical grounding pad.

Alternatively, a disposable coversheet may be designed to be used with a non-disposable, reusable, perinatal care device during a cesarean section surgery to provide an absorbent and protective barrier for the device.

This example of the invention relates to the use of a non-disposable perinatal care device when promoting skin to skin contact (SSC) between the mother and baby during and following a cesarean birth. The perinatal care device is a non-disposable, inflatable device that when used can safely and effectively support SSC for a mother and baby.

A large body of research supports early SSC as a precursor to successful initiation and duration of breastfeeding and improved well-being of the mother-baby dyad. The American Academy of Pediatrics has recommended that all healthy infants should be placed and remain in SSC with their mothers immediately after delivery until the first feeding occurs. However, in the case of cesarean births this generally implies a change of practice within the hospital setting.

Cesarean section is the most common surgical procedure for women in the United States. In 2013, there were approximately 4 million babies born in the US and of these 32.7% were by cesarean section (CDC, 2014). This translates into 1.3 million cesarean births each year with the majority of these births eligible for early skin to skin contact for the mother and baby while in the operating room.

For the hospital, providing SSC for their mothers and babies for cesarean birth may require many changes to their current practice. The hospital committed to providing safe and efficient SSC in the operating room may consider purchasing the perinatal care device(s) to more effectively provide this service for their mothers and babies. The number of perinatal care devices needed would depend largely upon the number of cesarean deliveries performed each year.

A cesarean birth results in large amounts of bodily fluids such as amniotic fluid and bleeding from the surgical site. The average intraoperative blood loss associated with cesarean delivery is approximately 1000 cc. The average volume of amniotic fluid is about 800 cc. Although there are surgical drapes on the market with pockets to help retain some or much of this fluid, these drapes are positioned over rather than underneath the mother allowing fluid to collect below her. As the perinatal care device is not disposable, it may require extensive cleaning following use, thus, taking it out of service for an undetermined amount of time.

Utilizing the disposable coversheet in conjunction with the perinatal care device may serve as a cost effective solution for minimizing the cleaning and usage turnover time for the perinatal care device. No other disposable sheet exists with the appropriate measurements to adequately cover and provide a protective barrier from bodily fluids for the perinatal care device.

An alternate embodiment of a perinatal care device consisting of a semi-circular wall portion which surrounds the mother's head, shoulders and arms, a pillow and a left lateral tilt pad and a shield portion that is placed over the mother's upper torso, thus providing a unitary design offering the mother and baby a safe and comfortable environment to promote skin to skin contact (SSC) during and following cesarean birth.

In an embodiment, the wall portion surrounds the pillow, which raises the mother's head or head and upper torso so the wall does not uncomfortably enclose the mother's face. This pillow is comprised of a separate inflatable chamber and once inflated prevents the baby from slipping past the mother's shoulders toward the base of the semi-circular wall near the mother's head.

In an embodiment, the distal ends of the wall portion referred to as the shield, with one portion located on the right side of the device and one on the left. The shield portions are positioned on each side of the mother's torso and when in use, are brought together, adjusted and fastened over the mother's sternum. When the two shield portions are connected together they may provide sufficient support for the mother to rest her elbows as she holds her baby on her chest. The semi-circular wall and the shield have a single, dedicated, inflatable chamber.

In an embodiment, the left lateral tilt pad consists of a separate inflatable chamber and is located underneath the mother's right upper torso and positioned at the level of her waist and upper buttock and is connected to the flat base panel.

In an embodiment, the flat base panel connects the semi-circular wall, pillow and left lateral tilt pad together to create one unitary device. This may be constructed by heat sealing or sewn in seams. The flat base panel, when positioned on the OR table, is located directly under the mothers torso.

An alternate method of warming may be the use of pre-warmed air supply pump technology when inflating this device, thus creating a warmed enclosure supporting a normothermic environment for the baby.

In addition, use of heat reflective technology, for example “reflective blankets” such as those made by Thermoflect™ could be placed over the infant to enhance the warming effects of pre-warmed cotton blankets which are currently used for the baby in many operating rooms.

When warmed blankets are placed over the infant's body, the design of the perinatal care device may aid in the reduction of heat loss experienced by the newly born baby by creating an enclosure around the mother and baby maintaining the warmth within the walls and the shield.

The device may be constructed using heat sealed or sewn seams to provide uniform structural strength while allowing bending at the junction of the wall and shield portion, wherein, both sides of the shield are flexible and angle toward the center when being fastened. The device may be constructed using a conductive fabric such a Lectrolite™ to control electrostatic discharge or may be made from a material such as a nonwoven, engineered fabric, so as to be disposable. If the device is constructed to be disposable, it may be incorporated with a disposable air transfer mattress so that the perinatal care device and the air transfer device are constructed as one unit. This unit may utilize the various methods of air inflation for the perinatal care device as seen in the non-disposable version, and as an example, could utilize an individual inflation port at the head of the device for inflation of the air transfer mattress portion of the unit.

In an embodiment, the perinatal care device may be inflated using a dial regulator attached to the air supply such as an existing dedicated pump associated with or included in the perinatal care device. The dial regulator would utilize a one or three way valve to allow for inflation of the selected chamber within the perinatal care device. A PSI regulator may be used to control the amount of air used to inflate each chamber. An alternate method of inflation would utilize a clamp on each of the tubes entering each chamber to be inflated. With the clamp in the opened position the pump could be used to inflate the selected chamber. With the clamp in the closed position the inflated chamber would remain inflated. A rapid deflation valve could be utilized for each inflatable chamber.

In an embodiment, the perinatal care device may be attached to an air transfer mattress using compatible fasteners and together may be used for transfer of the mother and baby from the operating table to the stretcher or bed.

In an embodiment, the perinatal care device may be designed to include a ramp feature as indicated in the previous patent application and would be inflated using a fourth air inlet, a fourth interior inflation tube and a fourth exterior inflation tube attached to the dial regulator for delivering the air supply from the pump.

The perinatal care device could have attachable adapters of various sizes that would be interchangeable to facilitate use of different air supply pumps.

### BRIEF DESCRIPTION OF DRAWINGS

The above and other aspects, features, and advantages of examples of the present invention will be more apparent from the following more particular description thereof, presented in conjunction with the following drawings wherein:

FIG. 1 is a side elevation view of an embodiment of an inflatable perinatal care device in an in-use condition;

FIG. 2 is a side elevation view of the device of FIG. 1 in an installed but uninflated condition;

FIG. 3 is a top view of the device of FIG. 1 in an uninflated condition;

FIG. 4 is a partially sectional side elevation view of the device of FIG. 1 and associated devices;

FIG. 5 is a plan view an inner liner forming part of the device of FIG. 1;

FIG. 6 is an axial sectional view of an embodiment of an air connector assembly for the device of FIG. 1;

FIG. 7 is a side elevation view of an alternative form of air connector; and

FIG. 8 is a detail view of an emergency deflation valve.

FIG. 9 is a side view of an embodiment of an alternate inflatable perinatal care device attached to a compatible air transfer mattress in an in-use condition;

FIG. 10 is a side elevation view of the device of FIG. 9 in an installed but uninflated condition;

FIG. 11 is a top view of the device of FIG. 9 in an uninflated condition;

FIG. 12 is a detail view of the air inlets for the device;

FIG. 13 is a top view of the device showing the interior and exterior inflation tubes;

FIG. 14 is a detail view of the pump regulator/exterior inflation tubes for the device of FIG. 9;

FIG. 15 is a view of the alternate form of exterior inflation tubes utilizing clamps;

FIG. 16 is a detailed view of the emergency deflation valves for the device;

Refer to FIG. 6 for axial section view of an embodiment of an air supply connector assembly for the device of FIG. 9;

Refer to FIG. 7. for the side elevation view of an alternative form of air supply connector;

Refer to FIG. 8 for an alternate view of an emergency deflation valve; and

FIG. 17 is a perspective view of the device and an associated device in the inflated condition;

FIG. 18 is an embodiment of a perinatal care device coversheet;

FIG. 19 is a detail drawing of the exterior inflation tubes exiting from the perinatal care device and coversheet; and

FIG. 20 is a drawing of a coversheet positioned on the perinatal care device as seen from the left side of the patient.

### DETAILED DESCRIPTION

### I. First Version

A better understanding of various features and advantages of the present methods and devices may be obtained by reference to the following detailed description and accompanying drawings, which set forth illustrative embodiments. Although these drawings depict embodiments of the contemplated methods and devices, they should not be construed as foreclosing alternative or equivalent embodiments apparent to those of ordinary skill in the subject art.

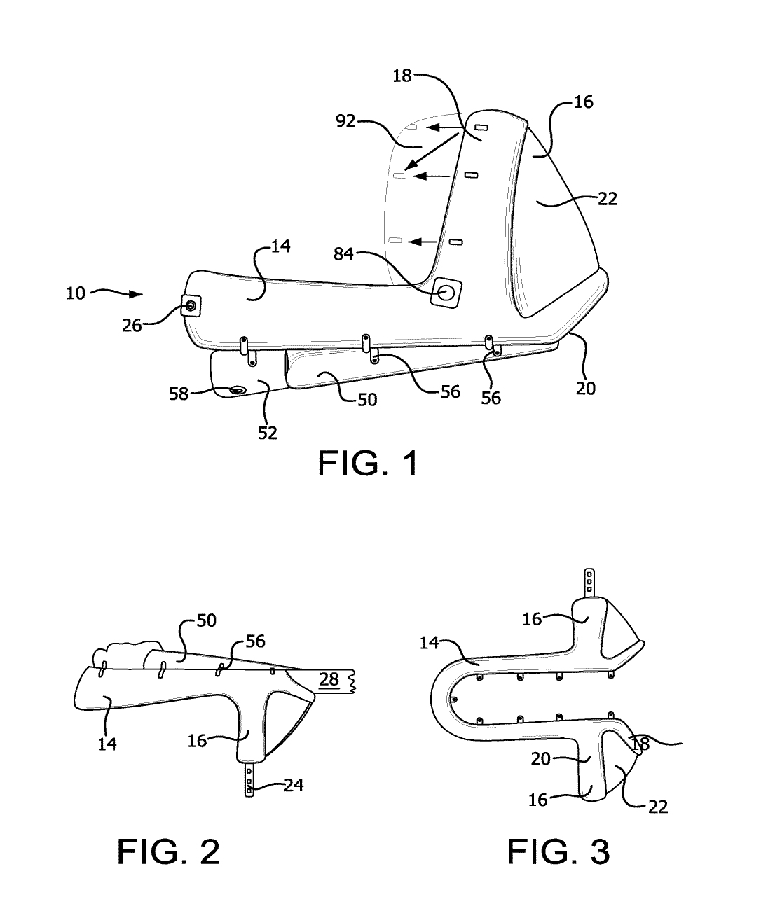

Referring to the drawings, and initially to FIGS. 1 to 3, one embodiment of a perinatal care device, indicated generally by the reference number 10, comprises a flat base panel 12 that in use lies on the operating table under the upper torso and head of the mother (not shown), a wall portion 14 that encircles the mother's head and shoulders, and two shield portions 16, one attached to either side of the base panel 12. The wall portion 14 is an inflatable tube that can be folded flat when uninflated, but that when inflated rises high enough to retain a baby against sliding sideways off the mother's thorax.

Each of the shield portions 16 comprises a rim tube 18, a body tube 20, and a membrane panel 22. When the shield is positioned over the mother, the body tubes 20 lie on the mother's upper thorax, meeting in the middle. The rim tubes 18 arch above the mother's sternum, meeting in the middle. Each membrane panel 22 extends between the body tube 20 and the rim tube 18, and the membrane panels meet in the middle. The two shield portions are joined in the middle by snaps, clasps, or other fastenings 24. The body tubes 20 and rim tubes 18 are inflatable, and when they are joined and inflated the shield is self-supporting. The body tubes 20, rim tubes 18, and wall portion 14 may be a single inflatable chamber that can be inflated through an air inlet 26. Alternatively, they may be separate chambers that either are separately inflated through inlets 26, or have internal valves to restrict the flow of air from one chamber to another.

As shown in FIG. 2, when the device 10 is uninflated, it can be hung over the head end of a standard hospital operating table 28, with the base panel 12 lying on the table 28, and the shield portions 16 and wall portion 14 hanging down. The device 10 is then effectively out of the way of the hospital personnel while they are placing and preparing the patient.

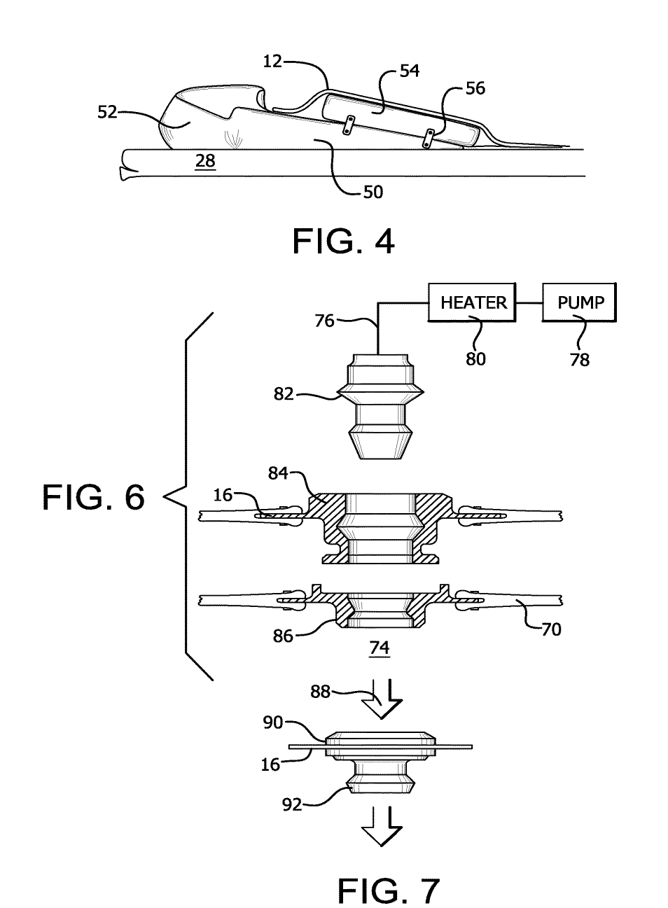

Referring now also to FIG. 4, the perinatal care device 10 may be used in combination with additional devices such as a ramp 50, and/or a pillow 52, and/or a “left lateral tilt” pad 54, which may also be inflatable. A ramp 50 is not preferred for obstetric use, although it may be beneficial for some other purposes. These additional devices 52, 54 may be unitary with the perinatal care device 10 or may be separate components. If they are separate components, the various devices are preferably provided with snaps or other fasteners 56 to hold them together in use, reducing the risk of problems because one device moves relative to another. The pillow 52 and “left lateral tilt” pad 54 may be placed on top of the base panel 12 of the perinatal care device 10, as shown in FIG. 2, or under the base panel 12, as shown in FIG. 1. Where the pillow 52 and/or “left lateral tilt” pad 54 is incorporated with the perinatal care device 10, it is preferably a separately inflatable and deflatable chamber.

As shown in the drawings, the additional devices 50, 52, 54 are inflatable devices provided with separate air inlets 58. If they are unitary with the perinatal care device 10, some or all of the devices may alternatively share a common air supply. Some or all of the additional devices 50, 52, 54 may alternatively be non-inflatable devices. Air to inflate the perinatal care device 10 and any additional devices 50, 52, 54 may be provided by an existing hospital compressed air supply, or by a dedicated pump associated with or included in perinatal care device 10. Because those devices are typically only inflated once for each surgical procedure and are then essentially static, a hand or foot pump may be sufficient, eliminating a hose or cable. However, an electric pump may be preferred in the interests of speed, especially if the perinatal care device 10 is being installed and inflated by a person who has many other responsibilities. The use of a hospital compressed air supply may require regulatory approval if the same air supply is also used for medical purposes.

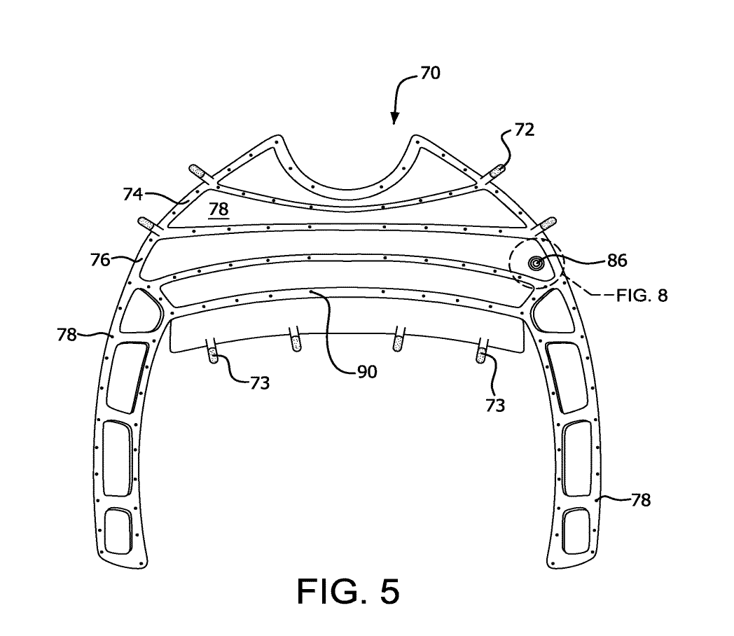

Referring now also to FIG. 5, the perinatal care device 10 includes an inner liner, indicated generally by the reference number 70. The inner liner 70 is placed inside the device after the two shield portions 16 have been joined by the fasteners 24, and is held in place by snaps or other fasteners 72, along the lower edge, near where the base panel meets the wall tubes 14 and body tubes 20, and fasteners 73 along the upper edge, along the rim tubes 18. The inner liner 70 comprises a manifold 74 in the form of a grid of air tubes 76 with membrane panels 78 between them. The air tubes 76 are perforated with air holes 78, allowing air to escape in calibrated amounts flowing in calibrated directions. In use, the manifold 74 is pressurized with warm air, and the air pressure holds the inner liner 70 against the inside of the shield portions 16.

Because the inner liner 70 is supported by the shield portions 16, the inner liner is not required to be very strong mechanically, and may be disposable. However, the inner liner may still be sufficiently strong to bridge a gap between the shield portions 16, allowing the perinatal care device 10 to fit round a mother of larger size than the nominal size of the device.

Referring now also to FIG. 6, the air supply to the manifold 74 may be provided by a hose 76 from a pump 78 through a heater 80. The pump 78 may be part of an existing hospital compressed air supply, or may be a dedicated pump for perinatal care device 10. At the end of the hose 76 is a connector 82, which locks into a port 84 passing through the wall of one shield portion 16 for mechanical security, and into a port 86 that opens into manifold 74. Locking between connector 82 and port 84 is desirable because, as explained above, shield portion 16 is stronger construction than inner liner 70. The risk of the lightly constructed liner 70 being damaged by a force applied to hose 76 is thus reduced. The use of a hospital compressed air supply may reduce noise and clutter in the operating area, but may entail additional regulatory requirements if the same air supply also supports medically sensitive functions elsewhere in the hospital.

Referring to FIG. 7, in an alternative form of air supply, a connector 88 on the hose 76 mates with the port 90 on the shield portion 16, and the port 90 includes a connector 92 that mates with the port 86 on the inner liner.

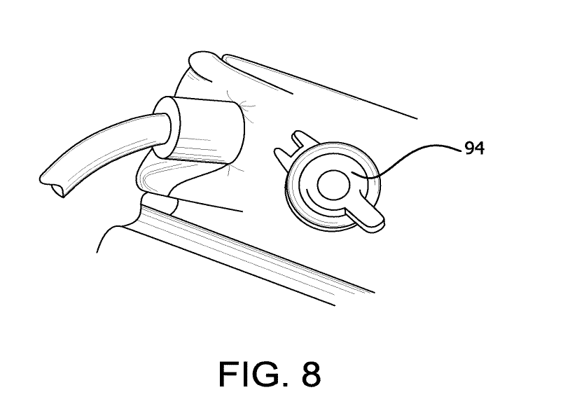

Each separately inflatable chamber of the device 10, including any pillow 52, left lateral tilt device 54, or other additional component, is provided with an emergency deflation valve 94 (see FIG. 8). The emergency deflation valves 94 may be of any suitable design. Their function is to allow all the air from the inflatable chamber to be released so as to deflate the chamber almost immediately if there is a medical need to lower the patient onto the flat, solid surface of the underlying mattress or operating table, or if there is an emergency in which the perinatal care device 10 needs to be removed quickly. A complex structure such as the manifold system of inner liner 70 may be provided with more than one emergency deflation valve 94.

In use, the perinatal care device 10 (not including the inner liner 70) and any desired additional devices 50, 52, 54 are assembled and placed in an uninflated condition over the head end of the operating table 28, as shown in FIG. 2. If the operating table 28, or a cover over the operating table, has suitable attachment points, the perinatal care device 10 may be attached to the table. Any desired warming pads, electrosurgical grounding pads, air cushion patient transfer pads, and other items may also be placed on the operating table at this time. Alternatively, if the patient is being brought in on a patient transfer pad, the perinatal care device 10 and additional devices 50, 52, 54 may be assembled on top of the patient transfer pad.

The mother is then placed on the operating table 28, on top of the base sheet 12 of the perinatal care device 10. The pillow 52 and/or left lateral tilt pad 54 may be inflated at this or any convenient later time, if they have an air inlet or air inlets 58 separate from the perinatal care device 10. Alternatively, the pillow 52 may be inflated before the mother's head is placed on the pillow. The inner liner 70 is then laid loosely over the mother's body, and the fasteners 72 around the outer edge of the inner liner are attached to the device 10.

The shield portions 16 are then folded up over the inner liner 70, and fastened in the middle with the fasteners 24. The inner liner 70 may be attached to the rim tubes 18 of the shield portions 16 by further fasteners 73 at this stage. To ensure a snug but not tight fit of the body tubes 20 around the mother's body, and a suitable height of the shield, the fasteners 24 are preferably adjustable, at least at the body tubes 20. The adjustment may allow an overlap between the shield portions 16 to fit a mother smaller than the nominal size of the perinatal care device 10, and/or a gap between the shield portions 16 to fit a mother larger than the nominal size of the perinatal care device 10. The inner liner 70, supported by the fasteners 24 themselves, can bridge a significant gap. It is presently believed that the fastening of the rim tubes 18 does not need to be adjustable.

The inflatable tubes 14, 18, 20 of the device are then inflated by supplying air through the port 26, to raise the walls 14 and the shield 16 to their desired height. The warm air pump 78 and heater 80 are then started, to supply warm air to the manifold 74 and, through the holes 78, to the space between the shield and the mother's upper torso.

As may be seen from FIGS. 1 and 5, the distribution of warm air can be controlled by the position of the holes 78 in the tubes 76. In particular, by placing holes 78 at different places around the circumference of the tubes 76, the direction of the warm air flow can be controlled. For example, the holes 90 in a tube 76 along the rim of the shield can produce a curtain of warm air, indicated by arrow 92 in FIG. 1, extending to the head end away from the shield 16. Thus, the area over the mother's upper thorax can be kept warm for the baby, without the rim of the shield itself overshadowing the mother's face uncomfortably.

The baby may then be placed in skin-to-skin contact with the mother, with both mother and baby being kept warm by the air from holes 78, 90, even while they are still in the cold operating room. If the mother is unable to maintain a secure grasp of the baby, and the baby starts to slide sideways, the baby is restrained by the walls 14, greatly reducing the risk of the baby falling off onto the floor, and removing the need for continuous supervision by a nurse. If the baby is given to the mother immediately after, for example, a cesarean delivery, the physical barrier formed by the perinatal care device 10 also protects the baby from undesirable contact with hard instruments, fluids, and other hazards involved in continuing surgical activity.

As an example of suitable dimensions, the device 10 may be about 30 inches (75 cm) long, measured from the top of the operating table 28. The side walls 14 may rise to a height of 8 inches (20 cm) above the table 28. The lowest portion of the shield, at the body tubes 20, may be adjustable to be 10, 12, or 14 inches (25, 30, or 35 cm) above the top of the operating table 28, and the top of the shield dome at the rim tubes 18 may be 17 inches (40 to 45 cm) above the operating table at its center. The rim 18 may begin about 11 inches (27.5 cm) from the top of the head in the lengthwise direction along the operating table. The left lateral tilt device 54 may be 4 inches (10 cm) wide and 3% inches (9 cm) high (around 14 inches (36 cm) in diameter), and 14 to 17 inches (35 to 44 cm) long, starting about 22 inches (55 cm) from the head end of the table. A device 10 with those dimensions would fit most mothers, but it may be desirable to provide larger and smaller sizes as well.

While the example of the invention has been disclosed with reference to certain preferred embodiments, numerous modifications, alterations, and changes to the described embodiments are possible without departing from the sphere and scope of the example of the invention, as defined in the appended claims and equivalents thereof.

For example, the device 10 has been described as a perinatal care device for use in permitting skin-to-skin contact between a newborn baby and his or her mother immediately after a cesarean birth. However, there may be other applications where it is desired to keep the head end of a patient warm and shielded during surgery on the abdomen or lower parts of the patient, and the use of the disclosed devices for such other applications is not excluded.

### II. Second Version

The perinatal device disposable coversheet is designed to provide an absorbent and protective barrier when used with the perinatal care device for cesarean birth. The disposable coversheet may consist of a nonwoven fabric such as SMS (100% polypropylene) for softness and drapability. It may be placed prior to or following inflation of the perinatal care device and secured with snaps, magnets, Velcro or by using an elastic gathering of the fabric allowing a snug fit over the contours of the perinatal care device or by any securing device determined to be suitable to hold the coversheet in place.

A mark(s) or other indication(s) on the fabric may be used to guide the user in the proper placement of the upper portion of the disposable coversheet. The top portion of the coversheet may be elastized to cover over the perinatal care devices' inflatable portions and the flat portion of the coversheet including the absorbent pad would continue lengthwise toward the foot of the operating table.

The disposable coversheet has an opening near the bottom of the headmost portion of the drape allowing the exterior inflation tubes of the perinatal care device to exit (where they eventually attach to the air supply pump). A strap with Velcro or some other means of reconnecting the two sides may be used to close the gap below the exit of the exterior inflation tubes.

As the perinatal care device is comprised of a semi-circular wall connected to a shield portion on the right side and left side of the device, the disposable coversheet is contoured and consists of two sleeves with an elastic opening on the end of each sleeve from which the shields are passed allowing them to be connected. When in use, the two ends of the shield are connected together in the center, above the mother's torso. Thus, the disposable coversheet protects each of the two connecting ends of the shield from becoming soiled.

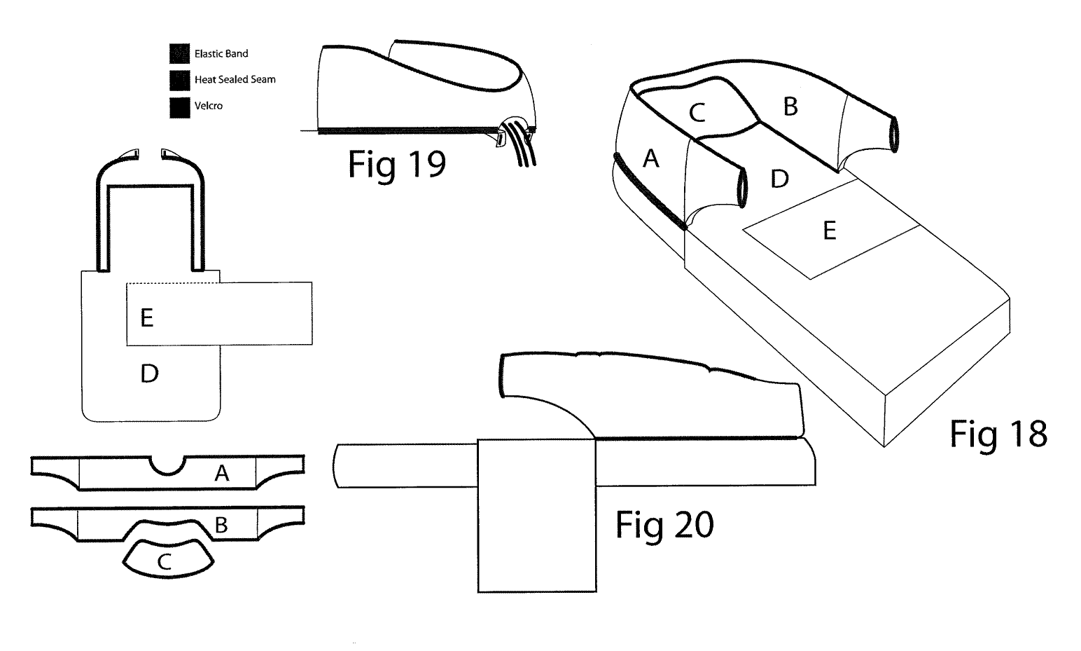

The mid portion of the coversheet provides absorbent protection from bodily fluids and is located just beneath the mother's lower back and buttock region as seen in FIG. 18 and FIG. 20, and is labeled E. The dimensions of this absorbent area are approximately 48 inches by 28 inches. The material for this portion of the coversheet will drape over the edges of the operating table and is considerably longer down from the mother's left side to allow for absorption of the bodily fluids which flow in this direction. This area of absorbent protection is perforated and easily removable when cleanup is desired prior to the transfer of the mother from the OR table. This absorbable layer will help protect the disposable coversheet, the underlying perinatal care device and the compatible air transfer mattress (if used) from bodily fluids. Additional layers of absorbable fabric could be utilized for this purpose.

In this same area, to the left side of the mother's buttocks, a strip of approximately 2″×24″ of thicker absorbent material to collect bodily fluids may be available and would also be removable. The absorbent material may be derived from a nonwoven polypropylene. For example, the strip would function much like a diaper or a sponge. This strip of absorbent material may be located in a plastic pocket or covered in clear plastic to prevent leaching of the collected bodily fluids toward the members of the surgical team.

The coversheet is long enough and wide enough to completely cover the perinatal care device and a compatible air transfer device (if used). As shown in FIG. 18.

### How to Use Second Version

The disposable coversheet is placed on the perinatal care device which is positioned on the operating room table. The coversheet may be placed before or after inflation of the perinatal care device. The tab located at the headmost portion of the coversheet is used for closing the gap in the area where the exterior inflation tubes of the perinatal care device exit. The contoured portion of the coversheet is placed over the pillow portion of the perinatal care device and then pulled over the wall portion. The two shield portions of the perinatal care device are pulled through the two contoured sleeves of the coversheet. Once the mother has been positioned on of the perinatal care device covered with the disposable coversheet the two covered shield portions are connected in the middle over the mother's sternum.

When the surgery has concluded, the mother and baby are prepared for transfer to the gurney or bed by tilting the mother and baby from left to right while removing the perforated absorbable pad portion of the coversheet and discarding. The portion of the disposable coversheet underneath the mother is now clean and dry. If being used, the air transfer mattress is inflated and the transfer is completed. The disposable cover may remain in place throughout the recovery period and until the mother is able to ambulate. It can be removed at that time along with the perinatal care device and air transfer mattress.

### Relationship Between the Components of Second Version

The disposable coversheet allows for a cost effective means for utilizing the perinatal care device and may increase the likelihood for the perinatal care device being used to its potential.

The disposable cover is easy to use and may replace the use of supplies currently used to set up an operating table for cesarean birth such as non-disposable linen draw sheets, disposable operating table sheets, absorbent pads, to name a few.

The coversheet provides a clean and comfortable surface for the mother during the intraoperative and postoperative period.

### Operation of Second Version

The design of the disposable coversheet allows for a conformed fit over the perinatal care device providing a barrier thereby protecting the perinatal care device from becoming soiled.

The design of the coversheet allows the surgical care team to easily remove the soiled absorbent pad from under the mother thus providing a clean, dry surface for increased maternal comfort.

The disposable coversheet allows for a cost effective means to utilize the perinatal care device and may improve the likelihood of the perinatal care device being used to its potential.

### How to Make the Second Version

The material used to make this product would need to meet similar regulations for flame resistance and anti-static control that may be required for medical supplies used in the operating room. An example of a material may be the SMS fabric that is nonwoven engineered with five strong fiber layers to create high barrier strength as a single use fabric.

The absorbent pad may be constructed of any of the many absorbent materials on the market that are used within the hospital environment and meet operating room specifications.

The fabric may have an impervious backing if needed for further protection.

The dimensions may be 52″ in width and×80″ in length.

The portion of the sheet covering the perinatal care device's pillow may be seamed or pleated to comfortably follow the contour of the pillow. The coversheet then rises up and over to surround the semicircular wall portion of the perinatal care device. The coversheet may be held in place with elastic gathering to secure it at the bottom exterior edges of the device. The coversheet follows the contour of the wall from the head of the operating table toward the foot of the table until it meets the shield portion of the device located at the end of the wall on each side of the device. At this junction the coversheet has two sleeves allowing one of the two ends of the shield to be pulled through each of the sleeves. Once each side of the shield has been pulled through the corresponding sleeve of the coversheet the two shields are joined together and the sleeves of the coversheet are pulled together to overlap.

The portion of the coversheet located just below the sleeves referred to as the “absorbent pad” would drape over the edge of the operating table toward the floor on the mother's left side. This same absorbent pad of the coversheet would cover the area under the mother's lower torso and buttocks. The absorbent pad is perforated along its upper edge located just below the sleeves.

### III. Third Version

A better understanding of various features and advantages of the present methods and devices may be obtained by reference to the following detailed description and accompanying drawings, which set forth illustrative embodiments. Although these drawings depict embodiments of the contemplated methods and devices, they should not be construed as foreclosing alternative or equivalent embodiments apparent to those of ordinary skill in the subject art.

Referring to the drawings, and initially to FIGS. 13 to 17, an alternate embodiment of a perinatal care device, indicated generally by the reference number 20, comprises a flat base panel 12 that in use lies on the operating table 28 under the head, neck and torso of the mother (not shown), a pillow 52 portion supporting the mother's head, a wall portion 14 that encircles the mother's head and shoulders, and two shield portions 16, one attached to either side of the base panel 12 and a left lateral tilt pad 54.

As shown in FIG. 17, the pillow supports the mother's head and upper shoulders and slopes slightly upward in a lateral direction from the center of the pillow to meet the wall portion. This slightly upward elevation eliminates a crevice at the junction between the pillow seam and the wall seam preventing the baby from possibly sliding down past the mother's shoulder and neck. The pillow is an inflatable chamber using the designated inflation inlet 32, as seen in FIG.12.

Referring to FIGS. 9 and 10, the wall portion 14 is an inflatable chamber that can be folded flat when uninflated, but that when inflated raises high enough to retain a baby against sliding sideways off the mother's chest. Each of the shield portions 16 previously consisted of the rim tube 18, body tube 20 and membrane panel 22. These tubes have been repositioned and comprise two single inflatable shields 16 which flex and arch above the mother's sternum, meeting in the middle. The two shield portions 16 are joined in the middle by snaps, clasps, or other fastenings 24. The wall portion 14 and the shield portion 16 are inflated as a single chamber using the designated inflation inlet 34 as seen in FIG. 16. The device is self-supporting when the wall and the two shield portions are fastened in the middle and the device is inflated.

Referring again to FIG. 9, the left lateral tilt pad 54 inflated elevates the right side of the mother's torso approximately 4.7 inches upward and toward the left side of the operating table. Tilting her toward the left side of the operating table is necessary to avoid compression of her major blood vessels by the unborn baby before and during the surgery. The left lateral tilt pad would be located under the right lumbar region, above the iliac crest and below the lower costal region of the mother's body to achieve a 12-15 degree tilt. The left lateral tilt pad is a separate inflatable chamber and is inflated using the designated inflation inlet 36, as seen in FIG. 16.

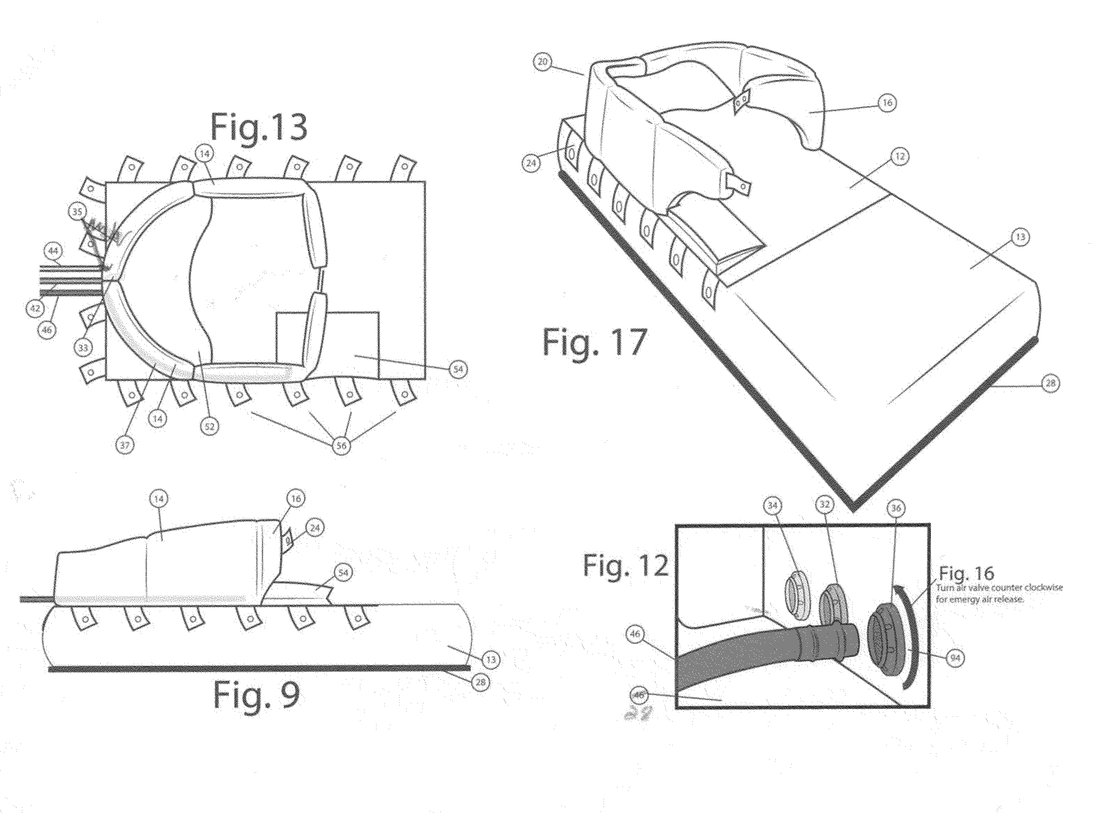

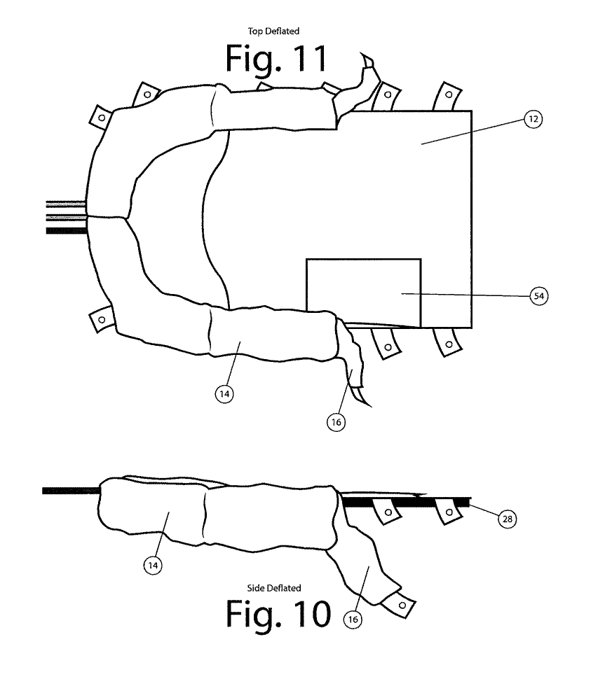

As shown in FIGS. 10 and 11, when the device 20 is uninflated, it can be hung over the head end of a standard hospital operating table 28, with the base panel 12 lying on the table 28, and the shield portions 16 and wall portion 14 hanging down.

As seen in FIG. 12, there are three air inlets 32, 34, and 36 which are located on the perinatal care device at just above the level of the operating table 28 and are installed within the device allowing each of the inflatable chambers to be securely connected into the appropriate corresponding exterior inflation tubes using a connector device 82 such as seen in FIG. 7 or 8. The exterior inflation tube 46 is shown as an example. The air inlets, 32, 34, and 36 are positioned on the centermost portion of the semicircular wall at the head of the device 20.

FIG. 13 demonstrates the configuration of the inflation tubes positioned within the interior of the device. Each of the three interior inflation tubes 33, 35, and 37 is attached to the inside of the corresponding air inlet 32, 34, and 36 (see FIG. 16). The interior inflation tube 33 transcends through the inflatable chamber of the wall 14 and attaches to the pillow 52 inflatable chamber. Next, the shortest of the three tubes, the interior inflation tube 35 attaches to the inflatable chamber of the wall 14 and shield 16. The longest of the three tubes, the interior inflation tube 37 transcends through the inflatable chamber of the wall 14 portion on the right side of the device, exiting the wall prior to the junction of the wall 14 and shield 16, entering and attaching to the inflatable chamber for the left lateral tilt pad 54. This allows the shield 16 on the right side of the device to flex and attach, uninhibited, at the center of the device when in use.

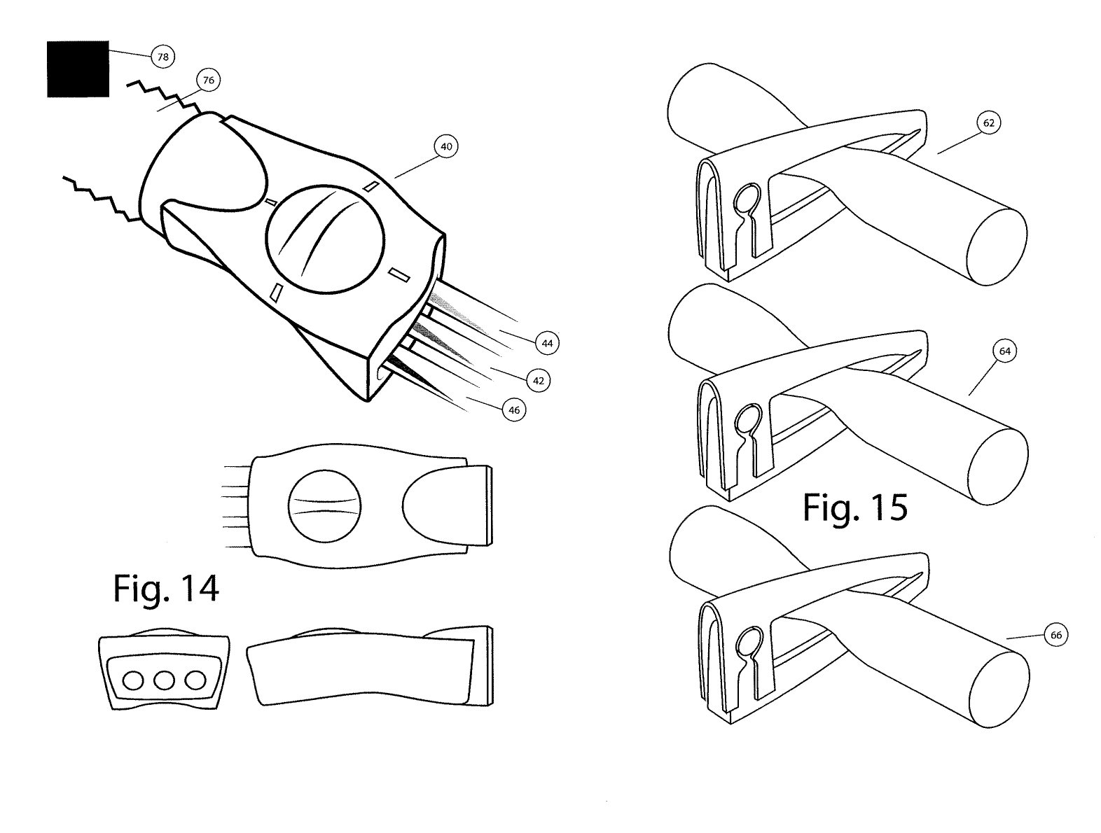

Referring to FIG. 14 the drawing details the pump regulator 40 and inflation tubing 42, 44, and 46 to be used for inflating the corresponding inflatable chamber through the air inlets 32, 34 and 36. The tubing may be color coded to correspond with the color of the air inlet or the dial may be labeled to indicate which air inlet to select. The opposing end of the pump regulator has the ability to connect to a designated pump hose for the air supply 76. A PSI regulator may be used to ensure the correct amount of air inflation to each of the chambers. The pump regulator may have a clip on the backside to enable it to attach to an OR table.

Referring to FIG. 15, is a drawing showing an alternative form of inflation for the device 20. Three separate tubes may be banded together with a release clamp on each tube, 62, 64, 66. Each of the three tubes would connect to one of the three air inlets 32, 34, or 36 located on the device 20 using a connector device 82. The opposing end of the tubes 62, 64, 66, may be banded together and placed within a compartment such as a larger plastic mold that would have the ability to connect to a designated pump hose for the air supply.

Referring to FIG. 16, if it is necessary to rapidly deflate the device the air inlet(s) 32, 34, 36 may also function as an emergency release valve(s). To deflate, disconnect the inflation tube(s) 42, 44, 46 from the air inlets or if using inflation tubes w/clamps 62, 64, 66, disconnect from the air inlets, grasp the air inlet and turn counter clockwise to release the one way valve allowing the selected chamber to rapidly deflate. See FIG. 8 as an alternate method of rapid deflation by using the emergency deflation valve 94.

Referring now also to FIG. 17, the perinatal care device 20 may be used in combination with additional devices such as an air transfer mattress generally referred to as 13. As a separate component, the air transfer device 13 is preferably provided with snaps or other fasteners 56 to attach to the perinatal care device. Using both devices together to assist in the transfer of the mother from the operating table to the gurney or bed may aid in protecting healthcare workers from injury.

In use, the perinatal care device 20 and if desired, a compatible air transfer mattress 13 (not shown) are assembled and placed in an uninflated condition on the operating table, as shown in FIGS. 10, 11. The pump 78 is attached to the perinatal care device using the pump regulator device 40 and associated exterior inflation tubes 42, 44, 46 or using the exterior inflation tubes with clamps 62, 64, 66. The pillow 52 and wall 14 and shield 16 portions are inflated to their desired height. The mother is placed on the operating table 28 on top of the base panel 12. On completion of the regional anesthetic, the mother is placed in the supine position and the left lateral tilt pad 54 is inflated. The two ends of the shield 16 are then brought together and attached with fasteners 24.

The baby may then be placed in skin to skin contact (SSC) with the mother, on her chest and covered with pre-warmed blankets or other such methods of preventing heat loss may be used. The mother can hold her baby safely without fear of losing her grip as the walls and shield prevent the baby from falling off. The nurse assigned to the care of the baby is then able to complete the assessments and other tasks without being required to be in constant physical contact with the baby as is currently necessary to prevent the infant from possibly slipping off the mother's chest and falling to the floor. The physical barrier formed by the shield 16 portion also protects the baby from undesirable contact with hard instruments, fluids, and other hazards involved in the continuing surgical activity.

The perinatal care device may remain inflated during transfer of the mother and baby when using a compatible air transfer mattress. The perinatal care device may be deflated at any time following the surgical procedure, transfer, or recovery of the mother.

With reference to a standard operating table using the measurements of 22 inches wide and 80 inches long, an example of suitable dimensions for the device 20 may be 40 inches in length when measured from the top of the operating table and 22-24 inches in width once inflated. The side wall may be 8 inches in height measured at the center of the head of the operating table and increasing in height from 8 inches to 12 inches as the wall extends lengthwise toward the shield portions (16) of the device. The height may increase to 13 inches at the junction of the wall (14) and the shield (16). The height of the shield may be adjustable to 10, 12, or 14 inches above the surface of the operating table when the two ends of the shield are attached together during use. The left lateral tilt positioning pad may be 17 inches in length and positioned 22-23 inches from the top of the perinatal care device ending at 39-40 inches measured from the top of the device. The left lateral tilt positioning pad also extends in width about 8-10 inches inward from the outer edge toward the center of the flat base panel (12) for the full 17 inch length. Once inflated, the pillow may be about 18 inches wide and 13-15 inches in length when measured from the top of the perinatal care device. Beginning from the top most portion, the pillow may be 4 inches in depth, tapering down to 1 inch in depth at the area where the mother's shoulders will rest.

What is claimed:

1. A patient care device comprising:
a shield that is configured to be placed over a patient's lower thorax, wherein the shield in an in-use configuration is generally in the shape of a part dome, with its rim extending towards the patient's head, and wherein a space between the shield and the mother's thorax is then sufficiently large for a newborn baby;
a manifold that in the in-use condition is in or on the shield and is provided with an inlet for air and air outlets to distribute said air to the space between the shield and the patient's thorax; and
a disposable cover sheet arranged over at least a portion of the shield.

2. The device of claim 1, further comprising a device for supplying warm air to said manifold through said inlet.

3. The device of claim 1, wherein said air outlets further comprise outlets at an upper rim of the shield positioned to emit jets of air angled obliquely, inwards and towards the patient's head.

4. The device of claim 1, further comprising wings that in the in-use condition extend from said shield towards the patient's shoulders, and are raised above a surface on which the patient and the device are placed.

5. The device of claim 4, wherein the wings are part of a continuous wall arranged to extend round the patient's head.

6. The device of claim 4, further comprising a ramp operative to raise the patient's head and shoulders above a level of a surface on which a lower part of the patient is lying.

7. The device of claim 6, wherein the wings are arranged to extend above the ramp.

8. The device of claim 1, comprising: a center section that is arranged to lie on a supporting surface under the patient's body; two side sections; and fasteners operative to connect the two side sections in a position folded together over the patient's body to form the shield.

9. The device of claim 8, wherein the fasteners are adjustable to fit patients of different sizes.

10. The device of claim 1, further comprising an inner liner removably positionable inside the shield and comprising said manifold.

11. The device of claim 10, wherein in use said inner liner is held in place inside said shield by allowing it to expand under the pressure of the air in the manifold.

12. The device of claim 1, wherein the shield is an inflatable device, supported by the pressure of air in compartments inside it in the in-use condition.
